(19) United States
(12) Patent Application Publication
(10) Pub. No.: US 2014/0274745 A1
(43) Pub. Date: Sep. 18, 2014

(54) METHOD FOR DETECTING MICRO-DELETION AND MICRO-REPETITION OF CHROMOSOME

(71) Applicants: Fang Chen, Shenzhen (CN); Xiaoyu Pan, Shenzhen (CN); Shengpei Chen, Shenzhen (CN); Xuchao Li, Shenzhen (CN); Hui Jiang, Shenzhen (CN); Xiuqing Zhang, Shenzhen (CN)

(72) Inventors: Fang Chen, Shenzhen (CN); Xiaoyu Pan, Shenzhen (CN); Shengpei Chen, Shenzhen (CN); Xuchao Li, Shenzhen (CN); Hui Jiang, Shenzhen (CN); Xiuqing Zhang, Shenzhen (CN)

(73) Assignee: BGI DIAGNOSIS CO., LTD., Shenzhen (CN)

(21) Appl. No.: 14/354,109

(22) PCT Filed: Oct. 28, 2011
(86) PCT No.: PCT/CN2011/001805

§ 371 Date: Apr. 24, 2014

(51) Int. Cl. C12Q 1/68; G06F 19/22

(52) U.S. Cl. CPC C12Q 1/6874; G06F 19/22
USPC 506/2; 702/19

(57) ABSTRACT

The present invention relates to the field of genomic mutation detection, and in particular, to the detection of the copy number variation (CNV) in cellular chromosomal DNA fragments. The present invention also relates to the detection of diseases related to the copy number variation in the cellular chromosomal DNA fragments.

### TECHNICAL FIELD

The present invention relates to the field of genomic mutation detection, and in particular, to the detection of the copy number variation (CNV) in cellular chromosomal DNA fragments. The present invention also relates to the detection of diseases related to the copy number variation in the cellular chromosomal DNA fragments.

### BACKGROUND ART

Chromosomal microdeletion/microduplication refers to the occurrence of a deletion or duplication of a length of 1.5 kb-10 Mb on a chromosome. Human chromosomal microdeletion/microduplication syndromes are a class of complex phenotype diseases caused by the occurrence of micro-fragment deletions or duplications (i.e., copy number variations in DNA fragments) on human chromosomes with a relatively high incidence in perinatal infants and neonatal infants, and can lead to serious diseases and abnormalities, e.g., congenital heart disease or heart malformation, serious growth retardation, appearance or limb malformation, etc. In addition, the microdeletion syndromes are also one of the main reasons causing mental retardation besides Down's syndrome and fragile X syndrome. [Knight SJL (ed): Genetics of Mental Retardation. Monogr Hum Genet. Basel, Karger, 2010, vol 18, 101-113]. In recent years, in the domestic and foreign statistics for the incidence of major birth defects, it is chromosomal microdeletions/microduplications related congenital heart disease, mental retardation, cerebral palsy and congenital deafness that are top-ranked. Common microdeletion syndromes include 22q11 microdeletion syndrome, cri du chat syndrome, Angelman syndrome, AZF deletion, etc.

With 22q11 microdeletion syndrome as an example, the syndrome is a class of clinical syndromes (including DiGeorge syndrome, velo-cardio-facial syndrome, conotruncal anomaly face syndrome, Cayler cardio facial syndrome, Opitz syndrome and a few other clinical syndromes with the same genetic basis) caused by the regional loss of heterozygosity of human chromosome 22q11.21-22q11.23, and the most common clinical manifestations of the disease include heart malformation, abnormal face, thymic hypoplasia, cleft palate and hypocalcemia; and in addition, a patient with the syndrome may also show physical and mental retardation, learning and cognitive difficulties, mental abnormalities and other manifestations, and the syndrome is the most common microdeletion syndrome in human, the incidence thereof being 1:4,000 (live births) and there being no significant difference in the incidence between men and women. [Drew L J, et al. The 22q11.2 microdeletion: Fifteen years of insights into the genetic and neural complexity of psychiatric disorders. Int J Dev Neurosci. 2010 Oct. 8.].

Although the incidence of each microdeletion syndrome is very low (https://decipher.sanger.ac.uk/syndromes), wherein the incidences of the relatively common 22q11 microdeletion syndrome, cri du chat syndrome, Angelman syndrome, Miller-Dieker syndrome, etc. are 1:4,000 (live births), 1:50,000, 1:10,000 and 1:12,000 respectively, due to the limitation by clinical detection techniques, a large number of patients with microdeletion syndromes cannot be detected in prenatal screening and prenatal diagnosis, and even when a reason is looked for retrospectively after the occurrence of typical clinical characterizations months or even years after the birth of an infant, the cause of the disease cannot be diagnosed also due to the limitation by the detection techniques. Because a radical cure cannot be effected for some types of microdeletion syndromes with the death within months or years after the birth, a heavy mental and economic burden is brought to the society and families. According to incomplete statistics, patients with “happy puppet syndrome” (i.e. Angelman syndrome) worldwide have reached 15 thousand. The numbers of patients with the other types of chromosomal microdeletion syndromes have also showed a trend of increase year by year. Thus, the detection of chromosomal microdeletions/microduplications performed progestationally on clinically suspected patients and parents with a related adverse pregnancy-labor history is conducive to providing genetic counseling and providing a basis for clinical decision; and the early prenatal diagnosis during pregnancy can effectively prevent the birth of an infant patient or provide a basis for providing a treatment approach in a targeted manner for an infant patient after birth [Bretelle F, et al. Prenatal and postnatal diagnosis of 22q11.2 deletion syndrome. Eur J Med Genet. 2010 November-December; 53(6): 367-370].

However, this class of diseases cannot be detected by routine clinical methods such as the chromosome karyotyping method (with a resolution of above 10 M) because of micro variations at the chromosome level [Malcolm S. Microdeletion and microduplication syndromes. Prenat Diagn. 1996 December; 16(13): 1213-9]. Currently, diagnostic methods for the microdeletion/microduplication syndromes mainly include high-resolution chromosome karyotyping, FISH (fluorescence in situ hybridization), Array CGH (comparative genomic hybridization), MLPA (multiplex ligation-dependent probe amplification technique), the PCR method and the like, and the use of these methods can detect chromosomal microdeletions/microduplications.

High-resolution chromosome karyotyping, which is a high-resolution banding technique that emerged after 1980s, adopts the cell synchronization method to obtain a large quantity of high-quality banding karyotypes of the late prophase or the early metaphase of mitosis, allows the number of bands of a single set of chromosomes to be increased to over several hundred, thereby improving the ability to recognize changes in the fine structure of the chromosomes, but the resolution thereof is only about 3-5 M. Although higher than routine chromosome karyotyping, the resolution of the method is insufficient to detect smaller microdeletion/microduplication variations at the chromosome level [Jorge J. Yunis, Jeffrey R. Sawyer and David W. Ball. The characterization of high-resolution G-banded chromosomes of man. Chromosoma. 1978 August, 67(4), 293-307].

FISH (fluorescence in situ hybridization) is a non-radioactive molecular cytogenetic technique developed in the late 1980s, the method is the gold standard for the detection of microdeletions/microduplications, and the method can effectively detect most of chromosomal deletions. The basic principle thereof is: if a target DNA on a chromosome or DNA fiber section to be tested is homologous and complementary to a used nucleic acid probe, the two undergo denaturation-annealing-renaturation and can form a hybrid of the target DNA and the nucleic acid probe. A certain species of nucleotide in the nucleic acid probe is labeled with a reporter molecule such as biotin and digoxin, and the immunochemical reaction between the reporter molecule and a specific fluorescein-labeled avidin can be used to perform qualitative, quantitative or relative location analysis on the DNA to be tested through a fluorescence detection system under a microscope. The advantages thereof are: a short experimental period, ability to get a result quickly, good specificity and accurate location. The resolution of FISH for metaphase chromosomes can reach 1-2 M, and the resolution of FISH for interphase chromosomes can reach 50 K, but the technique needs to design a probe to perform validation under the condition of known deletion sites, and is unsuitable for discovering a new microdeletion or duplication abnormality at the chromosomal level, and the price is expensive and there is a high requirement on the technical proficiency of an operator [Fluorescence in situ hybridization. Nature Methods, 2237 2238, 2005].

Array CGH (microarray-comparative genomic hybridization), a technique applied in the field of clinical cytogenetics in recent years, uses a specific DNA fragment as a target probe, immobilizes same on a carrier to form a microarray, and detects the DNA copy number variation through the hybridization of fluorescein-labeled DNA to be tested and reference DNA with the microarray. The resolution of Array CGH depends on the type and size of the designed probe and the distance thereof on the genome, and can theoretically detect 5 to 10 kb or even smaller DNA sequences, but the method is expensive in price and generally, does not cover all sites in the whole genome. Currently, diagnoses for chromosomal microdeletion syndromes have been more common in the literature [ACOG Committee Opinion No. 446: array comparative genomic hybridization in prenatal diagnosis. Obstetrics and Gynecology, 2009].

MLPA (multiplex ligation-dependent probe amplification technique) is a new technique developed in recent years for the qualitative and semi-quantitative analysis of a DNA sequence to be tested. Currently in clinical laboratories, the MLPA technique has been applied in the detection of Y chromosome microdeletions, 22q11.2 chromosome microdeletions and the like, the advantages are high efficiency, specificity, rapidness and simplicity and convenience, and the disadvantages are samples' susceptibility to contamination, unsuitability for the detection of an unknown type of point mutation and inability to detect the balanced chromosomal translocation [Wang Ke, et al., Detection of 22q11.2 chromosome microdeletion by MLPA technique. Proceedings of the Seventh National Cheilopalatognathus Academic Conference, 2009].

The PCR method is commonly used for the detection of Y chromosome microdeletions, e.g., the deletion of the male reproduction related AZF gene (AZFa, AZFb, AZFc) and the like on the Y chromosome is mostly detected by the PCR method. The PCR method can also be used for the validation of known chromosomal microdeletion sites. The method is simple, convenient and practicable, and the disadvantage is that the detection can only be aimed at known sites and the detection can merely be aimed at one site in a single run. A specific detection method needs to be combined with PCR reactions for a plurality of sites, so as to achieve the purpose of detection [Cong-yi Y U, et al. Multiplex PCR Screening of Y Chromosome Microdeletions in Azoospermic Patients. JOURNAL OF REPRODUCTION AND CONTRACEPTION. 2004, 15(4)].

It can be known from the combination of the above-mentioned content that currently, the existing limitations on the methods for detecting chromosomal microdeletions/microduplications mainly include low resolution, inability to cover the whole genome, low throughput and high cost. The development of a new method for detecting chromosomal microdeletions/microduplications which overcomes these limitations is urgently needed.

### SUMMARY OF THE INVENTION

With the continuous development of the high-throughput sequencing technique and the continuous reduction in the sequencing cost, the detection and analysis of chromosomal abnormalities by the high-throughput sequencing have been more and more widely applied. For solving the defects in the current methods for detecting chromosomal microdeletions/microduplications such as low resolutions, the present disclosure designs a high-throughput sequencing technique based method for detecting the DNA copy number variation and then detecting chromosomal microdeletions/microduplications. The method overcomes the disadvantages of low resolution, inability to cover the whole genome, low throughput and high cost in the several commonly used methods in the prior art, detects chromosomal microdeletions/microduplications on the whole-genome level, and not only can find and validate known sites for diseases, but also can explore and discover unknown sites, with high throughput, high specificity and accurate location. Through the detection of chromosomal microdeletions/microduplications, the detection of the chromosomal microdeletion/microduplication syndromes can be realized.

The present disclosure relates to a method for detecting the copy number variation (CNV) in cellular chromosomal DNA fragments, which includes the steps of:

a) randomly breaking genomic DNA molecules obtained from a subject and a normal subject to obtain DNA fragments, and sequencing said DNA fragments to obtain reads of sequencing;

b) aligning the DNA sequences determined in step a) to a genomic reference sequence of the species of said subject, locating the determined DNA sequences on the reference sequence, and only selecting and using reads with a unique position on the reference sequence to perform analysis;

c) seeking sites on the reference sequence which meet the following condition: a site with a difference in the copy number variation ratio on the two sides of the site compared with the alignment result of the normal sample, the steps being as follows:

i) for each site b on the reference sequence, forcing local windows on left and right sides thereof to contain w normal reads, i.e., to meet N(x_{L},b)=N(b,x_{R})=w, where N(x_{L},x_{R}) is the alignment number falling within the window (x_{L},x_{R}) for the normal sample;

ii) among these positions, screening sites which meet

$$b = {min}_{x} p ( \vert {D}_{x} ( {x}_{L} , {x}_{R} ) \vert ) ,$$

and excluding sites which meet Dᵢ(x_{L},x_{R})=0 and b−w<i<b+w, where D(x_{L},x_{R})=log(R(x_{L},x))−log(R(x,x_{R})) and

$$R ( {x}_{L} , {x}_{R} ) = \frac{T ( {x}_{L} , {x}_{R} ) / {a}_{T}}{N ( {x}_{L} , {x}_{R} ) / {a}_{N}} ,$$

where the numbers of reads of the normal sample and of reads of the sample to be tested which are uniquely aligned to the reference sequence are a_{N} and a_{T} respectively, and the numbers of reads which uniquely fall within the window (x_{L},x_{R}) are N(x_{L},x_{R}) and T(x_{L},x_{R}) respectively, and through the two-sided significance test for normal distribution on the test statistic D(x_{L},x_{R}), obtaining p(|D(x_{L},x_{R})|) for each site

iii) setting p_{bkp}, and repeating the above steps until all sites meeting p(|D(x_{L},x_{R})|)>p_{bkp} are obtained, so as to obtain a collection of candidate sites which is B^{c}, B^{c}={b₁, b₂, . . . , b_{N}};

where P_{bkp} can be set, for example, according to the data of the control sample, the minimum p(|D(x_{L},x_{R})|) is p_{bkp} when initial candidate sites are set as 10, 100, 1,000 or 10,000; p_{bkp} can also be selected through the following manner:

taking the normal sample as a sample to be tested, executing the aforementioned steps a) to ii) in c), filtering all p(|D(x_{L},x_{R})|) through false discovery rate control (FDR control), and taking the last p(|D(x_{L},x_{R})|) breaking an FDR threshold in post-filtration sites as p_{bkp}; the steps for the false discovery rate control being:

sorting datasets to be tested by significance (P value) in an ascending order to obtain their ranks (r);

performing the test from top to bottom until a stop at the last site k which meets

$${P}_{k} \leq \frac{{r}_{k}}{N} α ,$$

where Pₖ is the P value of the kth position, rₖ is the rank of the kth position, N is the total number of the sites, and α is the significance level, e.g. 0.01;

and retaining k and all sites before same, and removing false-positive sites after same;

d) for the collection of the candidate sites on the reference sequence obtained in step c) which is B^{c}, B^{x}={b₁, b₂, . . . , b_{N}}, the windows (bₖ₋₁, bₖ−1) and (bₖ,bₖ₊₁ existing on both sides of each site k, removing sites with a relatively small difference in the copy number variation ratio between the windows on the two sides, i.e., deleting the site k with the maximum p(|D_{b}_{ₖ}(bₖ₋₁,bₖ₊₁)|) each time, updating the p value of the merged interval (bₖ₋₁,bₖ₊₁), and through setting p_{merge}, repeating the step until all sites meet p(|D_{b}_{ₖ}(bₖ₋₁,bₖ₊₁)|)<p_{merge}, and the remaining sites being sites which meet the requirements needed to seek CNV, i.e., the breakpoints where the chromosomal copy number variation occurs being obtained;

where p_{emerge} can be set, for example, the maximum p(|D(x_{L},x_{R})|) is set as p_{merge} when the scale of the remaining sites is made to be ½, 1/10, 1/100 or 1/1,000 of the original one; p_{emerge} can also be selected through the following manner: taking the normal sample as a sample to be tested, executing the above-mentioned steps a) to d) to make the number of the candidate sites after merging become 1/2, 1/10, 1/100 or 1/1,000 of the initial number of sites, where the maximum p(|D(x_{L},x_{R})|) is selected as p_{merge}.

The present disclosure also relates to an analytical method for detecting a class of diseases which produce complex clinical phenotypic effects due to the copy number variation (CNV) in cellular chromosomal DNA fragments, and besides including the above-mentioned steps a)-d), said method also includes:

e) performing CNV analysis based on the breakpoints obtained in step d), and selecting sites where the CNV ratio of the sample to be tested relative to the normal sample is less than or equal to a detection threshold for microdeletions as microdeletion sites; and selecting sites where the CNV ratio of the sample to be tested relative to the normal sample is greater than or equal to a detection threshold for microduplications as microduplication sites,

where the detection threshold for microdeletions and the detection threshold for microduplications can be selected by a person skilled in the art according to the experience, for example, the detection threshold for microdeletions is 0.75 and the detection threshold for microduplications is 1.25;

f) performing basic gene annotation and functional analysis of genes involved in deletion parts on said microdeletion sites and/or microduplication sites compared with an existing CNV and disease database, and noting the type of the microdeletion syndrome disease.

For the specific technical flow of the embodiments of the present invention, see FIG. 1.

### Effect of the Invention

Compared with the current commonly used methods for detecting chromosomal microdeletions/microduplications (e.g., high-resolution chromosome karyotyping, FISH, Array CGH and the PCR method), the superiority of the present disclosure includes the following main points:

1) High resolution. In the present disclosure, the precision of the chromosomal CNV analysis can reach 100 kb, and the chromosomal microdeletions/microduplications can be detected effectively.

2) Being suitable for a wider data analysis, and increasing the utilization rate of memory devices. The algorithm is recompiled, the method for data processing is improved, the original SegSeq software is only suitable for 1-4× low depth sequencing data analysis, and the improved SegSeq can be used for data analysis of different sequencing depths of 1-30×.

3) Covering the whole genome. On the basis of the second-generation sequencing technique, the present disclosure can perform chromosomal CNV analysis on the scope of the whole genome, does not need to rely on known probes and the design of probes, and can discover new chromosomal abnormalities.

4) High throughput. On the basis of the high-throughput sequencing technique, the present disclosure can perform chromosomal CNV analysis in a high-throughput manner, and through the addition of different tag sequences to each sample, can analyze a large quantity of samples in a single run.

5) Low cost. With the continuous development of the sequencing technique and the continuous reduction in the sequencing cost, the cost of the chromosomal CNV analysis by the present disclosure is also decreasing continuously.

### DESCRIPTION OF THE DRAWINGS

FIG. 1 is a brief flow diagram of the chromosomal CNV analysis in the present disclosure.

FIG. 2 is a schematic flow diagram of the SeqSeq algorithm.

FIGS. 3A-C are digital chromosomal karyograms of sample 1-sample 3 with chromosomal duplications, deletions and normal regions as shown in the figures respectively, see Table 2 for corresponding positions and detailed information.

FIGS. 4A-C are digital chromosomal karyograms of sample 4-sample 6 with chromosomal duplications, deletions and normal regions as shown in the figures respectively, see Table 4 for corresponding positions and detailed information.

### PARTICULAR EMBODIMENTS

In the description and the claims of the present disclosure, reads refer to sequence fragments obtained by sequencing.

In the description and the claims of the present disclosure, a breakpoint refers to a demarcation point where the copy number variation occurs on a chromosome.

In the present disclosure, a genomic DNA obtained from a subject can be acquired from the blood, tissues or cells of a subject. Said blood can be from the peripheral blood of parents or the umbilical cord blood of a fetus; said tissues can be the placental tissue or the chorionic tissue; and said cells can be uncultured or cultured amniotic fluid cells and villus progenitor cells.

In the present disclosure, the genomic DNA can be acquired using the salting-out method, the column chromatography method, the magnetic bead method, the SDS method and other routine DNA extraction methods, preferably using the magnetic bead method. The so-called magnetic bead method refers to for bare DNA molecules obtained after the blood, tissues or cells undergo the action of a cell lysis solution and proteinase K, using specific magnetic beads to perform reversible affinity adsorption on the DNA molecules, and after proteins, lipids and other impurities are removed by washing with a rinsing liquid, eluting the DNA molecules from the magnetic beads with a purification liquid. The magnetic bead method can be performed according to the protocol provided by the manufacturer.

In the present disclosure, the treatment of randomly breaking DNA molecules can use enzyme digestion, atomization, ultrasound or the HydroShear method. Preferably, the ultrasound method is used, for example, for the AFA technique based S-series of the Covaris Corporation, when the sound energy/mechanical energy released by a sensor passes through a DNA sample, gas is dissolved to form bubbles. When the energy is removed, the bubbles burst and the ability to fracture DNA molecules is generated. Through setting a certain energy intensity and time interval and other conditions (the following are examples of breaking parameters: Duty cycle 20%, Intensity 10, cycles/Burst 1000, Time 60 s, Mode: power tracking), the DNA molecules can be broken into a certain range of sizes (for example, ranging from 200-800 bp). Please see the instruction provided by the manufacturer for the specific principle and method, and the DNA molecules are broken into fragments of a certain relatively concentrated size. In one embodiment of the present invention, the DNA molecules are broken into the size of about 500 bp.

In the present disclosure, the sequencing method used can be the high-throughput sequencing methods Illumina/Solexa, ABI/SOLiD and Roche454. The type of sequencing can be single-end sequencing and pair-end sequencing, and the sequencing length can be 50 bp, 90 bp or 100 bp. In one embodiment of the present invention, the sequencing platform is Illumina/Solexa, the type of sequencing is pair-end sequencing, and 100 bp sized DNA sequence molecules with a pair-end positional relationship are obtained.

In the present disclosure, the sequencing depth can be 1-30×, i.e., the total amount of data is 1-30 times the length of the human genome, for example, in one embodiment of the present invention, the sequencing depth is 2×, i.e., 2 times (6×10⁹ bp). The specific sequencing depth can be determined according to the size of detected chromosomal variation fragments, and the higher the sequencing depth is, the smaller the detected deletion and duplication fragments are.

When the DNA molecules to be tested are from a plurality of test samples, different tag sequences can be added to each sample to be used to distinguish the samples in the sequencing process [Micah Hamady, Jeffrey J Walker, J Kirk Harris et al. Error-correcting barcoded primers for pyrosequencing hundreds of samples in multiplex. Nature Methods, 2008, 5(3)], thereby realizing that the plurality of samples are sequenced simultaneously.

In the present disclosure, a genomic reference sequence can be from a public database. For example, a human genome sequence can be the human genome reference sequence in the NCBI database. In one embodiment of the present invention, said human genome sequence is the human genome reference sequence build 36 in the NCBI database (hg18; NCBI Build 36).

The sequence alignment can be performed through any sequence alignment program, for example, the Short Oligonucleotide Analysis Package (SOAP) and the BWA (Burrows-Wheeler Aligner) alignment that are available to a person skilled in the art, and the reads are aligned with the reference genome sequence to obtain the reads' positions on the reference genome. The sequence alignment can be performed using the default parameters provided by the program, or the parameters are selected by a person skilled in the art according to the requirements. In one embodiment of the present invention, the alignment software used is SOAPaligner/soap2.

In the present disclosure, what aligns the reads to the chromosomal sequence data is software like SOAP; and the software algorithm for the genomic copy number variation (CNV) is a Matlab script (group) developed by the Broad Institute, which is referred to as the Segseq software algorithm. See FIG. 2. Through data produced by the new-generation sequencing technique, by virtue of the comparison of a cancerous sample and a normal sample, it is able to calculate breakpoints of copy fragments and the copy number variation ratio (tumor-normal copy ratio), and at the same time, can estimate the corresponding P-value and other statistical data, and can detect CNV fragments of around 50 K at a low sequencing depth (10 M PE: 32,36 reads).

In the present disclosure, seeking breakpoints for CNV analysis for a sample to be tested, refers to using the improved Segseq software algorithm, taking a normal sample as a negative control, and seeking candidate sites in the sample to be tested where the difference in the copy number variation ratio on the two sides meets a certain requirement. Said seeking the breakpoints includes two steps: (1) initialization, with the purpose of selecting candidate points; and (2) repeating merging adjacent fragments, with the purpose of reducing the false positive rate.

The specific principle and the mathematical model are: on the premise that reads obtained by sequencing are random fragments from a genomic DNA, the number of reads falling in a region after alignment should obey a Poisson distribution. Assuming that the length of regions capable of being aligned in the whole genome is A (A=2.2×10⁹), the numbers of reads of a normal sample and of a sample to be tested that can be aligned to the reference sequence are a_{N} and a_{T} respectively, the numbers of reads that fall within the window (x_{L},x_{R}) are N(x_{L},x_{R}) and T(x_{L},x_{R}) respectively, and the size of the window is L=x_{R}−X_{L}+1, then N and T obey a Poisson distribution with a parameter of

$${λ}_{N} = \frac{{a}_{N} L}{A} and {λ}_{T} = \frac{{a}_{T} L}{A}$$

respectively, and λ_{T}=r×a×λ_{N}, a=a_{T}/a_{N}. The copy number variation ratio is defined as

$$R ( {x}_{L} , {x}_{R} ) = \frac{T ( {x}_{L} , {x}_{R} ) / {a}_{T}}{N ( {x}_{L} , {x}_{R} ) / {a}_{N}} ,$$

and under the condition of a very large sampling size, R(x_{L},x_{R}) is close to a logarithmic normal distribution. It is defined that D(x_{L},x_{R})=log(R(x_{L},x))−log(R(x,x_{R})), x_{L}<x<x_{R}. Then, since R(x_{L},x_{R}) is close to a logarithmic normal distribution, D(x_{L},x_{R}) obeys a normal distribution, so that the application of the two-sided P-value (p(|D(x_{L},x_{R})|>d)) can test whether the difference in the copy number variation ratio on the two sides of some site is significant.

The initialization in step (1) for seeking the breakpoints refers to the flow for initially selecting the candidate points. Specifically, for the position b on the reference sequence, the local windows on left and right sides thereof are forced to contain w normal reads, i.e., to meet N(x_{L},b)=N(b,x_{R})=w, and then among these positions, ones meeting

$$b = {min}_{x} p ( \vert {D}_{x} ( {x}_{L} , {x}_{R} ) \vert )$$

are added to a candidate sequence; but ones meeting Dᵢ(x_{L},x_{R})=0, b−w<i<b+w are excluded and not included in the candidate points. Through setting appropriate p_{bkp}, the above steps are repeated until p(|D(x_{L},x_{R})|)>p_{bkp} all to obtain an appropriate number of candidate points.

In the present disclosure, w can be any integer greater than 1, for example 5-5,000, preferably 10-2,000, more preferably 100-1,000, e.g. 300.

Repeating merging the adjacent fragments in step (2) for seeking the breakpoints, refers to that through the maximum likelihood processing, the adjacent fragments with a relatively small difference in the copy number variation ratio therebetween are made to be merged, thereby reducing the false positive rate. Specifically, assuming that the collection of the candidate points on the reference sequence obtained in step (1) is B^{c}, B^{c}={b₁, b₂, . . . , b_{N}}, and assuming that the windows on left and right sides of the candidate point k are (bₖ₋₁,bₖ−1) and (bₖ,bₖ₊₁) respectively, sites with a relatively small difference in the copy number variation ratio between the windows on the two sides are removed. That is, the site k with a maximum p(|D_{b}_{ₖ}(bₖ₋₁,bₖ₊₁)|) is deleted each time and the p value of the merged interval (bₖ₋₁, bₖ₋₁) is updated, and through setting p_{merge}, the step is repeated until all sites meet p(|D_{b}_{ₖ}(bₖ₋₁,bₖ₊₁)|)<p_{merge}, and then the remaining sites are sites meeting the requirements needed to seek CNV.

In the present disclosure, the CNV analysis after seeking the candidate points refers to according to empirical values of population data analysis in the field, taking a CNV ratio of a sample to be tested relative to a normal sample ≦0.75 and that ≧1.25 as detection thresholds for the chromosomal copy number variations respectively, with the case of CNV ratio ≦0.75 being a chromosomal deletion and the case of CNV ratio ≧1.25 being a chromosomal duplication. According to the analysis, microdeletion/microduplication results are obtained and a digital chromosomal karyogram is drawn.

A digital chromosomal karyotype is a technique for quantifying the DNA copy number variation on a genome, which lists short DNA sequences of specific sites on the whole genome separately. For example, for human chromosomes, drawing a chromosomal karyogram is usually arranging the chromosomes in a cell from the largest one (Chromosome 1) to the smallest one (Chromosome 22), with the sex chromosomes (X and/or Y) displayed at the end. This is an expression method commonly used in the field, and is within the competence scope of a person skilled in the art. For example, same can be performed with reference to the articles [Tian-Li Wang et al. Digital karyotyping. PNAS, 2002, vol. 99, no. 25, 16156-16161.] and [Henry Wood et al. Using next-generation sequencing for high resolution multiplex analysis of copy number variation from nanogram quantities of DNA from formalin-fixed paraffin-embedded specimens. Nucleic Acids Research, 2010, 38(14), doi: 10.1093/nar/gkq510.] or the examples of the present disclosure.

In the present disclosure, p_{bkp} therein can be set, for example, according to the data of the control sample, the minimum p(|D(x_{L},x_{R})|) is p_{bkp} when initial candidate sites are set as 10, 100, 1,000 or 10,000; p_{bkp} can also be selected through the following manner: taking the normal sample as a sample to be tested, executing the steps of the present disclosure to calculate p(|D(x_{L},x_{R})|), performing false discovery rate control (FDR control) on all p(|D(x_{L},x_{R})|), and taking the last p(|D(x_{L},x_{R})|) breaking an FDR threshold as p_{bkp}. For example, in the examples, different from cancer samples, default control samples (e.g., paracancerous ones) were not present in a population study, and therefore, we used the deep sequencing data of the data of the Yanhuang population (45 southern Han race+45 northern Han race) to compensate for resulting deficiencies. We took a mixed normal sample (only the data of the Yanhuang population except Yanhuang No. 1 are given herein) as a sample to be tested, executed the steps a) to ii) in c) in the method of the present disclosure respectively, performed false discovery rate control (FDR control) on all p(|D(x_{L},x_{R})|), and took the last p(|D(x_{L},x_{R})|) breaking the FDR threshold as p_{bkp}.

In the present disclosure, p_{merge} therein can be set, for example, the maximum p(|D(x_{L},x_{R})|) is set as p_{merge} when the scale of the remaining sites is made to be ½, 1/10, 1/100 or 1/1,000 of the original one; p_{merge} can also be selected through the following manner: taking the normal sample as a sample to be tested, executing the steps a) to d) in the method of the present disclosure to make the number of the candidate sites after merging become ½, 1/10, 1/100 or 1/1,000 of the initial number of sites, where the maximum p(|D(x_{L},x_{R})|) is selected as p_{merge}. For example, in the examples, because of the lack of default control samples (e.g., paracancerous ones), we could not select the threshold through the method of merging default controls. We executed the method of the present disclosure on the mixed normal sample (only the data of the Yanhuang population except Yanhuang No. 1 are given herein) until the step of merging, until the number of the candidate points in the collection of the candidate points became 1/100 of the initial one, where the maximum p(|D(x_{L},x_{R})|) was selected as p_{emerge} which was used in the subsequent analysis.

In the present disclosure, for a method for calculating the P value in the significance test for normal distribution, the methods well known in the field can be used, the P value can also be calculated through a large quantity of existing software algorithms, and these algorithms are available to a person skilled in the art.

In the present disclosure, an existing CNV and disease database refers to an existing database of information about the correlation between copy number variations and diseases. In one embodiment of the present invention, the database used refers to DECIPHER (https://decipher.sanger.ac.uk/syndromes), and the 58 microdeletion/microduplication syndromes listed in the database are all contents of clear relationships between deletion and duplication fragments and diseases.

In one embodiment of the present invention, a specific method for performing the chromosomal CNV analysis of the villus tissue includes the steps of:

1. DNA extraction and sequencing: after the extraction of villus tissue DNA according to an operation manual of a genomic DNA extraction kit by the magnetic bead method (e.g., Tiangen DP329), a library is constructed according to the standard library construction flow for Illumina/Solexa. In this process, the villus tissue DNA is randomly broken through the ultrasound method into DNA molecules concentrated at around 500 bp, adapters used for sequencing are added at both ends, different tag sequences (indexes) are added to each sample, so that the data of a plurality of samples can be distinguished in the data obtained in a single run of sequencing.

2. Alignment and statistics: the second-generation sequencing method Illumina/Solexa sequencing (other sequencing methods such as ABI/SOLiD can be used to achieve the same or similar effect) is used, DNA sequences of fragments of a certain size, i.e. reads, are obtained for each sample and same are SOAP-aligned with the standard human genome reference sequence in the NCBI database to obtain information about that the tested DNA sequences are located at the corresponding positions of the genome. For avoiding the disturbance to the CNV analysis caused by repeat sequences, only reads that are aligned with the human genome reference sequence uniquely (unique reads) are selected as valid data for the subsequent CNV analysis, and the number thereof a_{T} is counted.

3. Data analysis: a known normal sample is taken as a negative sample, through the CNV analysis based on the SegSeq algorithm, breakpoints needed for the CNV analysis are sought and the copy number variation ratio of the sample to be tested relative to the normal sample is calculated, and through setting certain detection thresholds, microdeletions/microduplications of the chromosomal fragments of the sample to be tested are judged, a digital chromosomal karyogram is drawn, and the annotation of corresponding genes is performed. The specific process is as follows:

1) Initialization. For a position b on one and the same chromosome, the parameter w is set to make the local windows on left and right sides thereof contain 300 normal reads, i.e., N(x_{L},b)=N(b,x_{R})=w=300. Among the positions of the reads of the sample to be tested, ones meeting

$$b = {min}_{x} p ( \vert {D}_{x} ( {x}_{L} , {x}_{R} ) \vert )$$

are added to the candidate sequence, and ones meeting Dᵢ(x_{L},x_{R})=0, b−w<i<b+w are excluded. A p_{bkp} related parameter is set as 1,000 to make the initialization flow output 1,000 candidate points. The above-mentioned step of exclusion and addition to the candidate sequence is repeated, until all p(|D_{L},x_{R})|)>p_{bkp}, and the collection B^{c}, B^{c}={b₁, b₂, . . . , b_{N}}, of the candidate points on the chromosome c, is output.

2) Repeating merging adjacent fragments. For the collection of the candidate points obtained by the initialization, assuming that the windows on left and right sides of the candidate point k are (bₖ₋₁,bₖ−1) and (bₖ,bₖ₊₁) respectively, a p_{merge} related parameter is set as 10 to make the repeated division flow output a result of at most 10 false positive fragments. Through repeating merging adjacent fragments with a relatively small difference in the copy number variation ratio there between until all p(|(D_{b}_{ₖ}(bₖ₋₁,bₖ₊₁)|)<p_{merge}, the final valid candidate points needed for the CNV analysis, i.e. breakpoints, are obtained.

3) CNV analysis. The above-mentioned final breakpoints are counted, and assuming that a window between two certain breakpoints is (x_{L},x_{R}), the CNV ratio of the sample to be tested relative to the normal sample

$$R ( {x}_{L} , {x}_{R} ) = \frac{T ( {x}_{L} , {x}_{R} ) / {a}_{T}}{N ( {x}_{L} , {x}_{R} ) / {a}_{N}}$$

is calculated. Said CNV ratio of ≦0.75 and that of ≧1.25 are taken as detection thresholds for deletions and duplications of chromosomal fragments respectively, and after microdeletion/microduplication results are obtained by analysis, a digital chromosomal karyogram is drawn and the gene annotation is performed.

The method of the present disclosure is suitable for the chromosomal CNV analysis of animals and human, particularly mammals, more particularly human.

For example, the chromosomal CNV analysis of a population applicable to the present disclosure is conducive to providing genetic counseling and providing a basis for clinical decision; and the preimplantation diagnosis or prenatal diagnosis can effectively prevent the birth of a patient infant. The population applicable to the present disclosure can be a population who have no abnormality in routine chromosomal karyotyping but have the following clinical manifestations:

1) females with multiple embryo damages or spontaneous abortions and spouses thereof;

2) females who have ever born malformation fetuses and spouses thereof;

3) male infertility patients with azoospermia or oligospermia;

4) male infertility patients with unknown causes;

The instances of the above-mentioned applicable population are only used to describe the present disclosure, and should not limit the scope of the present invention.

The following will illustrate the embodiments of the present invention in details in conjunction with examples, but a person skilled in the art will understand that the following examples are only used to describe the present invention, and should not be considered to limit the scope of the present invention. Those without indicated specific conditions in the examples are performed according to the routine conditions or the conditions recommended by the manufacturers. Reagents or instruments used without indicated manufacturers are all routine products available through the market. The manufacturer's article number of each reagent or kit is in the following brackets. The adapters and tag sequences used for sequencing are derived from the Multiplexing Sample Preparation Oligonutide Kit of the Illumina Corporation.

### Example 1

### Chromosomal CNV Analysis of 3 Tissues

1. DNA Extraction and Sequencing

According to the operation flow of the genomic DNA extraction kit by the magnetic bead method (TiangenDP329), DNA of 3 fetal tissue samples that have undergone chorionic centesis due to a high risk in prenatal screening (the value of risk being 1/9) and the case that the pregnant women themselves were balanced translocation carriers and having previously conceived one abnormal fetus (simply referred to as sample 1, sample 2 and sample 3 hereinafter, totally 2 villus tissue samples and 1 placental tissue sample) was extracted, and quantified with Qubit (Invitrogen, the Quant-iT™ dsDNA HS Assay Kit), and the total amount of the extracted DNA was about 500 ng.

The extracted tissue DNA was complete genomic DNA, and a library was constructed according to the standard library construction flow of Illumina/Solexa. In short, the adapters used for sequencing were added at both ends of DNA molecules which were broken to be concentrated at 500 bp, different tag sequences (indexes) were added to each sample which was then hybridized with complementary adapters on the surface of a chip (flowcell) to grow nucleic acid molecules in clusters under a certain condition, and then through double-end sequencing on Illumina Hiseq 2000, paired DNA fragment sequences of a length of 100 bp with a positional relationship were obtained.

Subsequently, after about 500 ng of DNA obtained from the above-mentioned tissues was randomly broken with Covaris S-series into 500 bp fragments, the modified standard flow of Illumina/Solexa was performed to construct a library, referring to the prior art for the specific flow (see the standard library construction instruction for Illumina/Solexa provided at http:www.illumina.com). The size of the DNA library and the size of inserted fragments were determined via 2100 Bioanalyzer (Agilent), and on-computer sequencing could be performed after precise quantification by QPCR. The total amount of data obtained finally for each sample was 6×10⁹ bp.

In the present example, the DNA samples obtained from the above-mentioned 3 tissues were operated according to instructions for Cluster Station and Hiseq 2000 (PE sequencing) published officially by Illumina/Solexa.

2. Alignment and Statistics

After undergoing said sequencing in step 1, each sample were distinguished according to said tag sequences, and DNA sequences of fragments of a certain size of about 500 bp, i.e. reads, were obtained. The alignment software SOAPaligner/soap2 was used to align the reads obtained by sequencing with the human genome reference sequence build 36 in the NCBI database (hg18; NCBI Build 36) to obtain information about that the tested DNA sequences were located at the corresponding positions of the genome. Only unique reads that were aligned with the human genome reference sequence uniquely were selected as valid data for the subsequent CNV analysis, and the number thereof a_{T} was counted.

In the present example, for the known normal sample, the Yanhuang genome DNA sample was selected as a negative sample control [Jun Wang, et al. The diploid genome sequence of an Asian individual. Nature. 2008 Nov. 6; 456(7218): 60-65].

The same amount of data as the samples to be tested were taken, and after standardization, the number of valid reads thereof a_{N} was counted, a_{N}=68750810. The numbers of valid reads a_{T}, of the above-mentioned sample 1, sample 2 and sample 3, were counted, being 25934245, 34164361 and 32085646, respectively.

3. Data Analysis

1) Initialization. The SegSeq algorithm was run, and for a position b on one chromosome, the parameter w=300 was set to make the local windows on left and right sides of the position b contain 300 normal reads, i.e., N(x_{L},b)=N(b,x_{R})=w=300. Among the positions of the reads of the samples to be tested, ones meeting

$$b = {min}_{x} p ( \vert {D}_{x} ( {x}_{L} , {x}_{R} ) \vert )$$

were added to the candidate sequence, and ones meeting Dₗ(x_{L},x_{R})=0, b−w<i<b+w were excluded. A p_{bkp} related parameter was set as 1,000 to make the initialization flow output 1,000 candidate points. The above-mentioned step of exclusion and addition to the candidate sequence was repeated, until all p(|D(x_{L},x_{R})|)>p_{bkp}, and the collection B^{c}, B^{c}={b₁, b₂, . . . b_{N}}, of the candidate points on the chromosome c, was output.

2) Repeating merging adjacent fragments. For the collection of the candidate points obtained by the initialization, assuming that the windows on left and right sides of the candidate point k were (bₖ₋₁,bₖ−1) and (bₖ,bₖ₊₁) respectively, a p_{merge} related parameter was set as 10 to make the repeated merging flow output a result of at most 10 false positive fragments. Sites with a relatively small difference in the copy number variation ratio between the windows on the two sides were removed, until all p(|D_{b}_{ₖ}(bₖ₋₁,bₖ₊₁)|)<p_{merge}, and the final valid breakpoints needed for the CNV analysis were obtained.

3) CNV analysis. The above-mentioned final breakpoints were counted, and assuming that a window between two certain breakpoints was (x_{L},x_{R}), the CNV ratios of the samples to be tested relative to the normal sample

$$R ( {x}_{L} , {x}_{R} ) = \frac{T ( {x}_{L} , {x}_{R} ) / {a}_{T}}{N ( {x}_{L} , {x}_{R} ) / {a}_{N}}$$

were calculated. Said CNV ratio of ≦0.75 and that of ≧1.25 were taken as detection thresholds for deletions and duplications of chromosomal fragments respectively, and after microdeletion/microduplication results were obtained by analysis, a digital chromosomal karyogram was drawn and compared with arrayCGH (The Fetal DNA Chip, http://www.fetalmedicine.hk/en/Fetal_DNA_Chip.asp). According to the DECIPHER database, the disease classification and the gene annotation were performed.

4) Outputting CNV analysis results and drawing the digital karyogram.

The copy numbers in the result of the negative control are all normal, and the CNV results of the 3 samples and the validation of the detection results and main genes are shown as in the following Tables 2 and 3, respectively.

TABLE 2

| | | | | | | Regions and |
|---|---|---|---|---|---|---|
| | | CNV starting | CNV ending | CNV | Judgment | bands |
| No. | Chromosome | point | point | size | result | involved |
| Sample | 5 | 1 | 36,862,895 | 36.9M | Deletion | 5p15.33→p13.2 |
| 1 | 18 | 38,986,536 | 76,117,152 | 37.1M | Duplication | 18q12.3→q23 |
| Sample | 13 | 97,076,671 | 106,514,142 | 9.4M | Deletion | 13q32.2→q33.3 |
| 2 | | | | | | |
| Sample | 2 | 230,295,360 | 242,427,661 | 12.1M | Duplication | 2q36.3→q37.3 |
| 3 | | | | | | |

TABLE 3

| | | | | Type of |
|---|---|---|---|---|
| | Regions | | | disease or |
| Sample | and | | | affected |
| No. | bands | arrayCGH result | Comparison | gene |
| Sample 1 | 5p15.33 | 5p15.3-p13.2(183931-36816731) × 1 | Consistent | Cri du |
| | →p13.2 | | | chat |
| | 18q12.3 | 18p12.3-q23(39086755-76067279) × 3 | Consistent | syndrome, |
| | →q23 | | | partial |
| | | | | trisomy |
| | | | | 18 |
| | | | | syndrome |
| Sample 2 | 13q32.2 | 13q32-q33.3(97091318-106466788) × 1 | Consistent | BIVM, |
| | →q33.3 | | | C13orf27, |
| | | | | KDELC1, |
| | | | | BIVM, |
| | | | | ERCC5 |
| Sample 3 | 2q36.3 | 2q36-q37.3(230369496-242444380) × 3 | Consistent | TRIP12, |
| | →q37.3 | | | SLC19A3, |
| | | | | PID1, |
| | | | | NYGGF4 |

It can be seen from the above-mentioned results that the chromosomal microdeletion and microduplication regions detected by high-throughput sequencing are consistent with the results of the prior arrayCGH (The Fetal DNA Chip, http://www.fetalmedicine.hk/en/Fetal_DNA_Chip.asp), and the specific digital karyograms can be seen in FIGS. 3A, 3B and 3C.

### Example 2

### Chromosomal CNV Analysis of Another 3 Villus Tissues

After 3 villus tissues (referred to as sample 4, sample 5 and sample 6 hereinafter) underwent the same treatment method and sequencing process as in Example 1, on-computer data were obtained, and the results were compared with the high-resolution karyotyping results.

In the data analysis process of the present example, the same as Example 1, for the known normal sample, the Yanhuang genome DNA sample was selected as a negative sample control, the same amount of data as the samples to be tested were taken, and after standardization, the number of valid reads thereof a_{N} was counted, a_{N}=68750810. The numbers of valid reads a_{T}, of the above-mentioned sample 4, sample 5 and sample 6, were counted, being 44797212, 44086450 and 45374254, respectively. The rest flow for data analysis and related parameter settings were all the same as those in Example 1, and finally, after microdeletion/microduplication results were obtained by analysis, a digital chromosomal karyogram was drawn and the gene annotation was performed.

The copy numbers in the result of the negative control are all normal, and the CNV results of the 3 samples and the validation of the detection results and main genes are shown as in the following Tables 4 and 5, respectively.

TABLE 4

| | | CNV | CNV | | | Regions |
|---|---|---|---|---|---|---|
| | | starting | ending | CNV | Judgment | and bands |
| | Chromosome | point | point | size | result | involved |
| Sample | 15 | 21,236,149 | 26,219,186 | 4.9M | Deletion | 15q11.2→q13.1 |
| 4 | | | | | | |
| Sample | 1 | 1 | 5,065,299 | 5M | Duplication | 1p36.33→p36.32 |
| 5 | | | | | | |
| Sample | 5 | 1 | 17,710,089 | 17.7M | Deletion | 5p15.33→p15.1 |
| 6 | | | | | | |

It can be seen from the above-mentioned results that for the 3 chorionic tissues, the chromosomal microdeletion and microduplication regions detected by high-throughput sequencing are consistent with the results of the prior arrayCGH (The Fetal DNA Chip, http://www.fetalmedicine.hk/en/Fetal_DNA_Chip.asp), and the specific digital karyograms can be seen in FIGS. 4A-C.

TABLE 5

| Sample | High-resolution karyotyping | | |
|---|---|---|---|
| No. | result | Comparison | Type of disease or affected gene |
| Sample 4 | 46, XX, del(15)(q11.2; q13.1) | Consistent | Happy puppet syndrome |
| | | | (Angelman syndrome) |
| Sample 5 | 46, XX, dup(1)p36.33; p36.32) | Consistent | 1p36 duplication syndrome |
| Sample 6 | 46, XX, del(5)p15.33; p15.1) | Consistent | Cri du chat syndrome |

It can be seen from the above-mentioned results that for the 3 chorionic tissues, the chromosomal microdeletion and microduplication regions detected by high-throughput sequencing are consistent with the results of the prior high-resolution karyotyping.

Although the particular embodiments of the present invention have been illustrated in details, a person skilled in the art will understand that according to all the teachings that have been disclosed, those details can be subjected to various modifications and substitutions, and these changes are all within the scope of protection of the present invention. All the scope of the present invention is given by the appended claims and any equivalent thereof.

1. A method for detecting the chromosomal copy number variation, comprising:
a) randomly breaking genomic DNA molecules obtained from a test sample and a normal sample to obtain DNA fragments, and sequencing said DNA fragments to obtain reads from sequencing;
b) aligning the DNA sequences determined in step a) to a genomic reference sequence of the species of said test and normal samples, locating the determined DNA sequences on the reference sequence, and only selecting and using reads with a unique position on the reference sequence to perform analysis;
c) seeking breakpoints on the reference sequence, wherein the breakpoint is a site with a difference in the copy number variation ratio on the two sides of the site compared with the alignment result of the normal sample, comprising:
i) for each site b on the reference sequence, forcing local windows on left and right sides thereof to contain w normal reads so that N(x_{L},b)=N(b,x_{R})=w, where N(x_{L},x_{R}) is the alignment number falling within the window (x_{L},x_{R}) for the normal sample, and w is an integer greater than 1;
ii) among these positions, screening sites which meet
$$b = {min}_{x} p ( \vert {D}_{x} ( {x}_{L} , {x}_{R} ) \vert ) ,$$
and excluding sites which meet Dᵢ(x_{L},x_{R})=0 and b−w<i<b+w, where D(x_{L},x_{R})=log(R(x_{L},x))−log(R(x,x_{R})) and
$$R ( {x}_{L} , {x}_{R} ) = \frac{T ( {x}_{L} , {x}_{R} ) / {a}_{T}}{N ( {x}_{L} , {x}_{R} ) / {a}_{N}} ,$$
where the numbers of reads of the normal sample and of reads of the test sample that are aligned with the reference sequence uniquely are a_{N} and a_{T} respectively, and the numbers of reads that fall within the window (x_{L},x_{R}) and are aligned with the reference sequence uniquely are N(x_{L},x_{R}) and T(x_{L},x_{R}) respectively, and through the two-sided significance test for normal distribution on the test statistic D(x_{L},x_{R}), obtaining p(|D(x_{L},x_{R})|) for each site;
iii) setting P_{bkp}, and repeating the above steps until all sites meeting p(|D(x_{L},x_{R})|)>p_{bkp} are obtained, so as to obtain a collection of candidate sites which is B^{c}B^{c}={b₁, b₂, . . . , b_{N}}, wherein p_{bkp} is selected by:
taking the normal sample as a sample to be tested, executing the aforementioned steps a) to ii) in c), filtering all p(|D(x_{L},x_{R})|) through false discovery rate (FDR) control, and
taking the last p(|D(x_{L},x_{R})|) breaking an FDR threshold in post-filtration sites as p_{bkp}; wherein the steps for the false discovery rate control comprise:
sorting datasets to be tested by significance (P value) in an ascending order to obtain their ranks (r);
performing the test from top to bottom until a stop at the last site k which meets
$${P}_{k} \leq \frac{{r}_{k}}{N} α$$
where Pₖ is the P value of the kth position, rₖ is the rank of the kth position, N is the total number of the sites, and α is the significance level, e.g. 0.01; and
retaining k and all sites before k, and removing false-positive sites after k;
d) for the collection of the candidate sites on the reference sequence obtained in step c which is B^{c}, B^{c}={b₁, b₂, . . . , b_{N}}, the windows (bₖ₋₁,bₖ−1) and (bₖ,bₖ₊₁) existing on both sides of each site k, removing sites with a relatively small difference in the copy number variation ratio between the windows on the two sides, i.e., deleting the site k with the maximum p(|D_{b}_{ₖ}(bₖ₋₁,bₖ₊₁)|) each time, updating the p value of the merged interval (bₖ₋₁,bₖ₊₁), and through setting p_{merge} and repeating the step until all sites meet p(|D_{b}_{ₖ}(bₖ₋₁,bₖ₊₁)|)<p_{merge}, so as to obtain the sites where the chromosomal copy number variation occurs.

2. The method according to claim 1, said w being an integer between 100-1,000.

3. (canceled)

4. The method according to claim 1, wherein
p_{merge} is the maximum p(|D(x_{L},x_{R})|) when the scale of the remaining sites is made to be ½, 1/10, 1/100 or 1/1,000 of the original one; or
p_{merge} is selected by:
taking the normal sample as a sample to be tested,
executing the above-mentioned steps a) to d) to make the number of the candidate sites after merging become ½, 1/10, 1/100 or 1/1,000 of the initial number of sites, and
selecting the maximum p(|D(x_{L},x_{R})|) as p_{merge}.

5. The method according to claim 1, after obtaining the sites where the chromosomal copy number variation occurs, further comprising,
e) performing analysis based on the sites, where the chromosomal copy number variation occurs, that are obtained in step d), selecting sites where the CNV ratio of the test sample relative to the normal sample is less than or equal to a detection threshold for microdeletions as microdeletion sites, and selecting sites where the CNV ratio of the test sample relative to the normal sample is greater than or equal to a detection threshold for microduplications as microduplication sites; and
f) performing gene annotation and functional analysis on said microdeletion sites and/or microduplication sites compared with an existing CNV and disease database, and noting the type of the chromosomal microdeletion and/or microduplication syndrome disease.

6. The method according to claim 5, said detection threshold for microdeletions being 0.75 and said detection threshold for microduplications being 1.25.

7. The method according to claim 1, said samples being derived from cells, blood or tissues.

8. The method according to claim 1, wherein randomly breaking genomic DNA molecules of the test and normal samples of step a) comprises chemical or physical fracture.

9. The method according to claim 1, wherein sequencing the DNA fragments of step a) comprises using a high-throughput sequencing technique.

10. The method according to claim 1, a range of the sequencing depth adopted in said step of sequencing the DNA fragments being 1-30×.

11. The method according to claim 5, further comprising: drawing a digital chromosomal karyogram, said digital chromosomal karyogram being drawn according to the values of the copy number variation ratios.

12. The method according to claim 8, wherein the chemical or physical fracture is performed using enzyme digestion breaking, or breaking by atomization, ultrasound or the HydroShear method.

13. The method according to claim 9, wherein the high-throughput sequence technique comprises Illumina/Solexa, ABI/SOLiD or Roche/454 sequencing.
